(19) United States
(12) Patent Application Publication
(10) Pub. No.: US 2022/0280463 A1
(43) Pub. Date: Sep. 8, 2022

(54) PHARMACEUTICAL FORMULATIONS COMPRISING DICLOFENAC

(71) Applicant: FTF PHARMA PRIVATE LIMITED, Ahmedabad (IN)

(72) Inventors: Jayanta Kumar MANDAL, Ahmedabad Gujarat (IN); Malay PATEL, Ahmedabad Gujarat (IN); Rutul PATEL, Vadnagar, Gujarat (IN)

(73) Assignee: FTF PHARMA PRIVATE LIMITED, Ahmedabad (IN)

(21) Appl. No.: 17/639,724

(22) PCT Filed: Sep. 9, 2020
(86) PCT No.: PCT/IB2020/058358

§ 371 Date: Mar. 2, 2022

(30) Foreign Application Priority Data
Sep. 9, 2019 (IN) 201921036219

(51) Int. Cl. A61K 31/196; A61K 9/00; A61K 47/02; A61K 47/20

(52) U.S. Cl. CPC A61K 31/196; A61K 9/0019; A61K 47/02; A61K 47/20

(57) ABSTRACT

Conventional injectable formulations of Diclofenac are known to have excipients which cause irritation at the site of injection and are painful. Further, such conventional formulations also cause thrombophlebitis. Other injectable formulations of Diclofenac known in the prior art also contain excipients which are tissue irritants and may cause toxicity when administered through intravenous route. The cyclodextrin compounds used in Dyloject® may cause problems while elimination in the renal compromised patients. The present invention therefore provides injectable Diclofenac formulations which do not cause irritation and pain at the site of injection. Further, the formulations of the present invention do not include cyclodextrins, therefore can also be administered to the renal compromised patients.

### FIELD OF THE INVENTION

The present invention is related to the field of pharmaceutical formulation technology. More particularly, the present invention is directed to the injectable pharmaceutical formulations of Diclofenac or its salts which overcome prior known problems such as irritation and pain at site of injection, thrombophlebitis and non-suitability for certain patient population. The formulations of the present invention are ready to use and are free or substantially free of cyclodextrin compounds.

### BACKGROUND OF THE INVENTION

Diclofenac is a non-steroidal anti-inflammatory drug indicated for use in adults for the management of mild to moderate pain and management of moderate to severe pain alone or in combination with opioid analgesics. In the year 2014, the United States Food and Drugs Administration (USFDA) approved Dyloject® as a single use vial containing 37.5 mg/ml Diclofenac sodium. Dyloject® is a clear, colorless, aqueous, nonpyrogenic sterile solution containing 37.5 mg of Diclofenac sodium (34.8 mg of Diclofenac), 333 mg of hydroxypropyl betadex (HPβCD), 5 mg of monothioglycerol, sodium hydroxide and/or hydrochloric acid for pH adjustment and water for injection. For the treatment of acute pain, the recommended dose of Dyloject® is 37.5 mg administered by intravenous bolus injection over 15 seconds every 6 hours as needed, maximum daily dose is 150 mg.

US 20050238674, incorporated herein by reference, discloses stable parenteral aqueous solution comprising either (a) diclofenac or a pharmaceutically acceptable diclofenac salt and a cyclodextrin, or (b) an inclusion complex of diclofenac or a pharmaceutically acceptable diclofenac salt and a cyclodextrin, or a mixture of (a) and (b), the solution containing: diclofenac or diclofenac salt; cyclodextrin; and an antioxidant selected from monothioglycerol, or a combination of ethylene diamine tetra-acetic acid and N-acetyl-cysteine, wherein the cyclodextrin is 2-hydroxypropylbeta-cyclodextrin.

US 20070232567, incorporated herein by reference, discloses a pharmaceutical composition comprising: (a) a dosage of a non-steroidal anti-inflammatory drug (NSAID) effective to induce analgesia an anti-inflammatory effect, or an anti-pyretic effect and (b) a beta-cyclodextrin compound; wherein the dosage of the NSAID compound is less than the minimum approved dose for the route of administration.

U.S. Pat. No. 7,423,028, incorporated herein by reference, disclose an injectable pharmaceutical composition in the form of an aqueous solution, comprising sodium diclofenac at a concentration of 75 mg/ml and hydroxypropyl-β-cyclodextrin, wherein said composition further comprises at least a polysorbate in an amount ranging between 0.01 and 0.06% by weight with respect to the total volume of the solution.

U.S. Pat. No. 8,946,292, incorporated herein by reference, discloses a method of providing analgesia to a subject in need thereof, which method comprises parenterally administering to the subject a pharmaceutical composition, wherein the composition comprises: (a) from about 18.75 mg to about 37.5 mg of a diclofenac compound; and (b) a beta-cyclodextrin compound; wherein the dosage of the diclofenac compound is less than about 1.3 mg/kg per day; and wherein the method achieves at least about 82% of maximum observable total pain relief assessed by a Visual Analog Scale.

U.S. Pat. No. 8,809,393, incorporated herein by reference, discloses an injectable formulation of water-soluble salts of diclofenac in Glycofurol and benzyl alcohol and claimed that intramuscular administration causes significantly less pain at the site of injection. However, Glycofurol is known as a tissue irritant while Benzyl alcohol causes thrombophlebitis.

U.S. Pat. No. 9,211,251, incorporated herein by reference, discloses an injectable preparation comprising: (A) at least one water-soluble salt of diclofenac (75 mg); (B) a single co-solvent selected from the group consisting of: a monohydric alcohol (4% to 25% v/v) selected from the group consisting of benzyl alcohol and ethyl alcohol, a polyhydric alcohol (27% to 45% v/v) selected from the group consisting of propylene glycol, polyethylene glycols having a molecular weight of between 300 to 600 Dalton, glycerin, and 1,3-butylene glycol, and glycofurol (18% to 35% v/v), in combination with water; (C) an alkali selected from the group consisting of sodium hydroxide and potassium hydroxide; and (D) a buffer system selected from the group consisting of phosphate buffer and bicarbonate buffer; wherein the viscosity of the preparation ranges from 1.50 to 4.7 centipoise (CPS); the final volume of the injectable solution is approximately 1 ml; the pH of the injectable solution is maintained at between 8 and 9 by said alkali and said buffer system; and the injectable solution is capable of being administered by the intradeltoid, intragluteal route, and/or slow intravenous route. However, Glycofurol is known as a tissue irritant while Benzyl alcohol causes thrombophlebitis.

US 20110275717, incorporated herein by reference, discloses a formulation containing salt of Diclofenac, at least one polyoxyalkylene ester of a hydroxyl fatty acid, water, and a co-solvent. This composition contains fatty acid derivative and they require special carriers.

US 20140187635, incorporated herein by reference, discloses a composition comprising a combination of water-soluble salts of Diclofenac in Transcutol and Benzyl alcohol. However, Transcutol is known as a tissue irritant while Benzyl alcohol causes thrombophlebitis.

WO 2016170401, incorporated herein by reference, discloses an injectable preparation comprising 75 mg/ml to 100 mg/ml of diclofenac sodium or therapeutically equivalent amounts of water soluble salts of diclofenac in a solvent system comprising two or more solubilizers in an amount ranging from 0.01 to 40% w/v and water and optionally one or more antioxidant(s) in an amount ranging from 0.10 to 0.5% w/v and/or one or more buffering agent (s) in an amount ranging from 0.05 to 1.0% w/v, wherein the pH of the preparation is maintained at 8-9. Here, solubilizers are selected from the group consisting of hydroxypropylbeta-cyclodextrin and polysorbate 80. The antioxidant is selected from the group consisting of monothioglycerol, sodium bisulphate and sodium metabisulphate. Buffering agent is selected from the group consisting of potassium dihydrogen phosphate, phosphate buffer and bicarbonate buffer.

US 20180271815, incorporated herein by reference, discloses storage stable aqueous injectable solution comprising Diclofenac and polyvinylpyrrolidone. The compositions disclosed in US 20180271815 further comprise one or more antioxidants, one or more surfactants and/or one or more pH adjusting agents. Here, antioxidant selected is monothioglycerol, surfactant selected is polysorbate 80 and pH adjusting agent selected is sodium hydroxide and/or hydrochloric acid. The pH of the compositions disclosed therein is between about 7.0 and about 10.0.

WO 2018142313, incorporated herein by reference, discloses a stable aqueous injectable composition of a therapeutically effective amount of diclofenac and/or pharmaceutically acceptable salts thereof, which is substantially free of oily components, cyclodextrins and benzyl alcohol. The compositions disclosed in WO 2018142313 further comprise co-solvents/solubilizers, a polar aprotic solvent, alkalizer, water and optionally other excipients. Preferred co-solvent/solubilizer is ethylene oxide/propylene oxide copolymer such as Pluronic® P 94 and Pluronic® F 68 (Poloxamer 188), a polyethoxylated castor oil such as Cremophor® EL, an ethoxylated polysorbate such as polysorbate 80 or alternatively a polyethylene hydroxystearate such as polyethylene hydroxystearate-660 or combination thereof. Preferred polar aprotic solvent is 1-methyl-2-pyrrolidone, 1,3-dimethyl-2-imidazolidinone, dimethylacetamide, dimethyl sulfoxide, acetone, tetrahydrofuran, 1,4-dioxane, acetonitrile, dimethyl formamide, propylene carbonate or combination thereof, particularly preferred polar aprotic solvent includes N,N-dimethylacetamide. Preferred alkalizer is selected from meglumine, sodium carbonate, potassium carbonate, calcium carbonate, magnesium oxide, calcium hydroxide, sodium hydroxide, potassium hydroxide, diethanolamine, potassium bicarbonate, potassium citrate, sodium borate, sodium citrate and triethanolamine.

The compositions disclosed in WO 2018142313 further comprise an antioxidant and chelating agent. Preferred antioxidant is butylated hydroxytoluene, butylated hydroxyanisole, tert-butyl-hydroquinone, 4-hydroxymethy-1-2,6-di-tert-butylphenol, 2,4,5-trihydroxybutyrophenone, alkylgallates, propyl gallate, octyl gallate, dodecyl gallate, ethoxyquin, gallic acid, nordihydroguaiaretic acid, glycine, ascorbic acid, fatty acid esters of ascorbic acid such as ascorbyl palmitate and ascorbyl stearate, and salts of ascorbic acid such as sodium, calcium, or potassium ascorbate; erythorbic acid, L-carnitine, thioglycerol, monothioglycerol, acetyl L-carnitine, thioglycolic acid, N-acetyl cysteine, cysteine, glutathione, methionine, tartaric acid, citric acid, fumaric acid, glycolic acid, oxalic acid, succinic acid, ellagic acid, malic acid, maleic acid, tocopherols such as delta tocopherol, alpha tocopherol; lipoic acid, thiolated polymers such as polymethacrylic-SH, carboxy methylcellulose-cysteine, polycarbophil-cysteine, beta-carotene, carotenoids, flavonoids, flavones, isoflavones, flavanones, catechins, anthocyanidins, chalcones, sulfites such as potassium sulfite, sodium metabisulfite, sodium sulfite, sodium thiosulfate and sodium bisulfite or a combination thereof. Preferred chelating agent is ethylene diaminetetraacetic acid (EDTA), deferoxannine, desferrioxannine B, dithiocarb sodium, penicillamine, pentetate calcium, a sodium salt of pentetic acid, succimer, trientine, nitrilotriacetic acid, diethylenetriaminepentaacetic acid, trans-diaminocyclohexanetetraacetic acid (DCTA), dihydroethylglycine, bis(anninoethyl)glycolether-N,N,N′,N′-tetraacetic acid, iminodiacetic acid, poly(aspartic acid), citric acid, tartaric acid, fumaric acid, succinic acid, glycolic acid, lactic acid, oxalic acid, malic acid, lecithin or any salt thereof, and the like or a combination thereof. The pH of the compositions disclosed in WO 2018142313 is between 8.1 and 9.0.

Excipients which are included in the earlier known Diclofenac formulations have some limitations for usage. For example, it is observed that elimination of cyclodextrin is a problem in renal compromised patient. Propylene glycol, glycofurol, transcutol etc. are tissue irritants and cause irritation at injection site. Further, propylene glycol in the formulation makes the injection painful. Benzyl alcohol causes thrombophlebitis. Intravenous (IV) use of polysorbates 80 should also be careful as they are known to cause toxicity when administered by IV route.

Diclofenac shows the property of cyclizing in an acid environment to give the corresponding indolinone. In order to obtain stabilisation of the open form, it is salified with some bases. U.S. Pat. No. 4,948,805, incorporated herein by reference, discloses the preparation of the salt of diclofenac with hydroxyethylpyrrolidine. Such a salt is soluble in water and allowed the commercialization of aqueous pharmaceutical forms of diclofenac. However, U.S. Pat. No. 4,948,805 does not disclose whether aqueous injectable formulations of Diclofenac using hydroxyethylpyrrolidine salt can be prepared or not.

US 20110275717, as discussed above, discloses a formulation containing salt of Diclofenac, at least one polyoxyalkylene ester of a hydroxyl fatty acid, water, and a co-solvent. This composition contains fatty acid derivative and they require special carriers. Though US 20110275717 mentions N-(2-hydroxyethyl)pyrrolidine as one of the organic amine, this document certainly does not teach aqueous injectable formulations of Diclofenac epolamine, i.e. N-(2-hydroxyethyl)pyrrolidine salt of Diclofenac.

The need therefore still exits for such Diclofenac parenteral formulations which can overcome above mentioned problems. The inventors of the present application have now surprisingly identified that the aqueous Diclofenac formulations even in the absence of cyclodextrins such as hydroxypropylbeta-cyclodextrin, solvents/co-solvents such as propylene glycol, polyethylene glycol, benzyl alcohol, glycofurol, transcutol etc. and other excipients, for example, surfactants such as polysorbates are stable and the active ingredient Diclofenac does not precipitate out from the aqueous solution when stored at different storage conditions after longer period of time.

### Objects of the Invention

An object of the present invention is to provide injectable formulations comprising Diclofenac or its pharmaceutically acceptable salt which are ready to use and can be administered to the patients in need thereof as such without prior reconstitution and/or dilution.

A yet another object of the present invention is to provide injectable formulations of Diclofenac which do not cause tissue irritation or irritation at the site of injection and which do not cause pain at the site of injection.

A yet another object of the present invention is to provide injectable formulations of Diclofenac which are suitable for all types of patient populations.

A yet another object of the present invention is to provide aqueous injectable formulations of Diclofenac in which the active ingredient Diclofenac or its salt remain in dissolved state even after prolonged time when stored under different storage conditions.

A yet another object of the present invention is to provide storage stable injectable formulations of Diclofenac or its salts.

A yet another object of the present invention is to provide liquid injectable Diclofenac formulations in the form of aqueous solutions comprising Diclofenac or its pharmaceutically acceptable salt such as epolamine and one or more pharmaceutically acceptable excipients. The injectable formulations of Diclofenac epolamine have advantages disclosed herein the present specification.

A yet another object of the present invention is to provide injectable Diclofenac formulations which are free or substantially free of cyclodextrins such as hydroxypropylbeta-cyclodextrin and organic solvents such as propylene glycol, polyethylene glycols & benzyl alcohol.

A yet another object of the present invention is to provide process for the preparation of the injectable Diclofenac formulations disclosed herein.

A yet another object of the present invention is to use the formulations of the present invention in adults for the management of mild to moderate pain and management of moderate to severe pain alone or in combination with opioid analgesics.

### DETAILED DESCRIPTION OF THE INVENTION

Diclofenac is a non-steroidal anti-inflammatory drug (“NSAID”) with analgesic, anti-inflammatory and anti-pyretic activity, most commonly used for treatment of acute and chronic pain and inflammation such as Musculoskeletal and joint disorders such as osteoarthritis, rheumatoid arthritis, and ankylosing spondylitis, management of post-operative pain, in both parenteral and oral dosage forms. It has also been used for the fever management. It is also used for the prevention of intraoperative miosis occurred during cataract extraction, for the treatment of inflammation after surgery or accidental trauma, and for the relief of ocular signs and symptoms of seasonal allergic conjunctivitis. Diclofenac is mainly used in form of sodium or potassium salts and act by the inhibition of arachidonic acid cyclooxygenase system, lipooxygenase pathway, arachidonic acid release resulting in decreased production of prostaglandins and thromboxanes, leukotrienes and reduction of arachidonic acid, respectively (Martindale, 2002). When given by oral route, dose is 100-200 mg/day, while by parenteral route, dose range from 75-150 mg/day given by either infusion or in divided doses. Toxicity of parenteral and oral forms are well established, includes gastrointestinal haemorrhagic, hepatic, renal, cardiovascular and allergic adverse events. Conventional formulations of Diclofenac Sodium injection are limited to intramuscular administration which causes substantial pain at the site. This limitation has been arising primarily due to the physicochemical properties of drug.

Due to poor aqueous solubility of the Sodium salt of Diclofenac, it has a high tendency to crystallize from aqueous and organic solutions. Potent solubilizing co solvents, such as Macrogols and Benzyl alcohol have been used to achieve physically stable solutions containing at least 75 mg/3 ml of Diclofenac Sodium which would allow intra-muscular (IM) administration of the desired dose. However, these co-solvents have an unfavourable intravenous safety profile and are associated with venous sequelae, high haemolytic and sensitizing (Reed, K. W. et al, J. Par. Sci. Technol. 39 (2) (1985) 64-68). Low solubility of Diclofenac salts has limited the parenteral use to IM and/or slow intravenous (IV) administration of diluted (100-500 ml diluent) product.

Further, these conventional formulations of diclofenac sodium contain high amounts of propylene glycol, which is known to cause irritation at injection site. It has been reported that the aqueous solution of 2% propylene glycol is isosmotic with serum causes 100% haemolysis of erythrocytes in 45 min. (Martindale, the Extra Pharmacopoeia 28th Edition). Also, high pH is required for solubilisation of Diclofenac Sodium and along with the hyper-osmolar nature of the formulation it contributes to the discomfort at the site of the injection when administered intramuscularly.

However, many attempts have been made to eliminate propylene glycol from the formulation to minimize pain at the site of the injection and improve the solubility of Diclofenac Sodium and then administer for treatment of various pathological conditions.

The N-(2-hydroxyethyl)pyrrolidine salt of Diclofenac also known as epolamine salt is a water soluble salt of Diclofenac. However, epolamine salt is used so far for the preparation of external preparations of Diclofenac such as patches, gels, lotions etc. The literature survey for Diclofenac formulations does not indicate use of epolamine salt of Diclofenac for the preparation of injectable or parenteral formulations.

In order to find out the solution, the present inventors have now developed Diclofenac injectable formulations which overcome aforementioned problems associated with the conventional Diclofenac formulations and Diclofenac formulations known in the prior art.

In one embodiment, the present invention provides injectable Diclofenac formulations which are ready to use and can be administered directly to the patients in need thereof without prior reconstitution and/or dilution. These types of ready to use formulations save time and the patients can be benefited by the required dose instantly.

In another embodiment, the injectable Diclofenac formulations of the present invention are aqueous solutions in which the active ingredient Diclofenac or its pharmaceutically acceptable salts remain in the dissolved state for prolonged time when stored under different storage conditions.

In another embodiment, the injectable Diclofenac formulations of the present invention do not cause tissue irritation and pain at the site of injection. In another embodiment, the formulations of the present invention are suitable for all types of patient populations.

In another embodiment, the injectable Diclofenac formulations of the present invention comprise Diclofenac or its pharmaceutically acceptable salt and an antioxidant. In some of the embodiments, the injectable Diclofenac formulations of the present invention further comprise one or more pharmaceutically acceptable excipients selected from the group of a tonicity adjusting agent, a buffering agent, a pH adjusting agent, a preservative (anti-microbial agent), a chelating agent and the like or any combinations thereof. The formulations of the present invention comprise water (e.g. water for injection) as vehicle.

In some of the embodiments, the injectable Diclofenac formulations comprise Diclofenac or its pharmaceutically acceptable salt, an antioxidant, a tonicity adjusting agent, a pH adjusting agent and water for injection.

In some of the embodiments, the formulations of the present invention are free or substantially free of cyclodextrins such as hydroxypropylbeta-cyclodextrin, organic solvents/co-solvents such as propylene glycol, polyethylene glycols, benzyl alcohol etc. and surfactants such as polysorbates.

In some embodiments, the present invention provides injectable formulations of Diclofenac epolamine and one or more pharmaceutically acceptable excipients. In some embodiments, the injectable formulations of Diclofenac epolamine are aqueous formulations. In some embodiments, one or more pharmaceutically acceptable excipients selected from the group consisting of an antioxidant, a tonicity adjusting agent, a buffering agent, a pH adjusting agent, a preservative (anti-microbial agent), a chelating agent and the like or any combination thereof. In some embodiments, injectable formulations of Diclofenac epolamine are free or substantially free of cyclodextrin compounds such as hydroxypropylbeta-cyclodextrin, organic solvents such as propylene glycol, polyethylene glycols, benzyl alcohol and the like and surfactants such as polysorbates.

As used herein, the term “free or substantially free” means that the formulations of the present invention either do not include those excipients or optionally comprise one or more of those excipients in such a concentration which does not produce the adverse effects for which they are known. Therefore, in some embodiments, the injectable formulations of Diclofenac epolamine optionally comprise one or more excipients selected from a solubilizing agent, a solvent/co-solvent, a surfactant, a stabilizing agent and the like or any combination thereof.

In some of the embodiments, the pH of the formulations of the present invention ranges between about 6.0 and about 11.0. In some embodiments, the pH of the formulations of the present invention ranges between about 7.0 and about 10.0. In some of the embodiments, the pH of the formulations of the present invention ranges between about 8.0 and about 9.0.

Antioxidants can be compounds that can reduce a drug that has been oxidized, or compounds that are more readily oxidized than the agents they are to protect (oxygen scavengers). Mixtures of chelating agents and antioxidants are often used because there appears to be a synergistic effect. This occurs because many of the agents act at differing steps in the oxidative process. The antioxidants when used in the compositions of the present invention include, but are not limited to, butylated hydroxytoluene, butylated hydroxyanisole, tert-butyl-hydroquinone, 4-hydroxymethyl-2,6-di-tert-butylphenol, 2,4,5-trihydroxybutyrophenone, alkylgallates, propyl gallate, octyl gallate, dodecyl gallate, ethoxyquin, gallic acid, nordihydroguaiaretic acid, glycine, ascorbic acid, fatty acid esters of ascorbic acid such as ascorbyl palmitate and ascorbyl stearate, and salts of ascorbic acid such as sodium, calcium, or potassium ascorbate; erythorbic acid, L-carnitine, monothioglycerol, acetyl L-carnitine, thioglycolic acid, N-acetyl cysteine, cysteine, glutathione, methionine, tartaric acid, citric acid, fumaric acid, glycolic acid, oxalic acid, succinic acid, ellagic acid, malic acid, maleic acid, tocopherols such as, but not limited to, delta tocopherol, alpha tocopherol; lipoic acid, thiolated polymers such as, but not limited to, polymethacrylic-SH, carboxy methylcellulose-cysteine, polycarbophil-cysteine, beta-carotene, carotenoids, flavonoids, flavones, isoflavones, flavanones, catechins, anthocyanidins, chalcones, sulfites, including but not limited to potassium sulfite, sodium metabisulfite, sodium sulfite, sodium thiosulfate and sodium bisulfite or a combination thereof may be employed. The use of an antioxidant in the form of monothioglycerol (0.1% to 1.0% w/v) has been found effectively stabilizes the solution preventing the formation of particulate matter at elevated temperature in pre-fillable syringes, ampoules and vials.

Chelating agents when used in the compositions of the present invention include, but are not limited to, ethylene diaminetetraacetic acid (EDTA), deferoxannine, desferrioxannine B, dithiocarb sodium, penicillamine, pentetate calcium, a sodium salt of pentetic acid, succimer, trientine, nitrilotriacetic acid, diethylenetriaminepentaacetic acid, trans-diaminocyclohexanetetraacetic acid (DCTA), dihydroethylglycine, bis(anninoethyl)glycolether-N,N,N′,N′-tetraacetic acid, iminodiacetic acid, poly(aspartic acid), citric acid, tartaric acid, fumaric acid, succinic acid, glycolic acid, lactic acid, oxalic acid, malic acid, lecithin or any salt thereof, and the like or a combination thereof may be employed.

Typically, tonicity adjusting agents are used to adjust the osmolality of the pharmaceutical compositions to bring it closer to the osmotic pressure of body fluids, such as blood or plasma. In some embodiments the tonicity of the formulation can be modified by adjusting the concentration of buffer and/or other components present in the formulation.

Provided that the compositions are physiologically compatible, the compositions do not require any particular osmolality. Thus, the compositions can be hypotonic, isotonic or hypertonic. Typically, the pharmaceutical compositions have a tonicity between about 250 to about 350 mOsm/kg. In some of the alternative embodiments, the formulations of the present invention are isotonic, i.e., in the range of 270-328 mOsm/kg. However, the formulations may have a tonicity in the range of 250-350 mOsm/kg. Therefore, the formulations may be either slightly hypotonic, 250-269 mOsm/kg, or slightly hypertonic, 329-350 mOsm/kg.

Suitable tonicity adjusting agents for use in the pharmaceutical compositions include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts. The quantity of the tonicity adjusting agent in the formulation can be expressed in mg/ml or in g/L. In typical embodiments, the tonicity adjusting agent(s) is present from about 1 mg/ml to about 90 mg/ml. Thus, the pharmaceutical compositions can comprise one or more tonicity adjusting agents at about 1-5 mg/ml, at about 5-10 mg/ml, at about 10-15 mg/ml, at about 15-25 mg/ml, at about 25-50 mg/ml, at about 50-60 mg/ml, at about 60-70 mg/ml, at about 70-80 mg/ml, and at about 80 to 90 mg/ml, as well as combinations of the above ranges.

Alternatively, the tonicity adjusting agent concentration is measured in weight/volume percent. In typical embodiments, the tonicity adjusting agent(s) is present from about 0.1% to about 10%. For example, suitable tonicity adjusting agent concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8%, from about 0.8% to about 0.9%, from about 0.9% to about 1%, from about 1% to about 2%, from about 2% to about 3%, from about 3% to about 4%, from about 4% to about 5%, from about 5% to about 6%, from about 6% to about 7%, from about 7% to about 8%, from about 8% to about 9%, and from about 9% to about 10%, as well as combinations of the above ranges.

In some embodiments, the tonicity adjusting agent is dextrose. Typically, the concentration of dextrose suitable for use in the pharmaceutical compositions is between about 2.5% (w/v) to about 7.5%. By way of example, suitable dextrose concentrations include, but are not limited to, from about 2.5% to about 3%, from about 3% to about 3.5%, from about 3.5% to about 4% (which is equivalent to about 40 mg/ml), from about 4% to about 4.5%, from about 4.5% to about 5% (which is equivalent to about 50 mg/ml), from about 5% to about 5.5%, from about 5.5% to about 6% (which is equivalent to about 60 mg/ml), from about 6% to about 6.5%, from about 6.5% to about 7%, as well as combinations of the above ranges.

In some embodiments, the tonicity adjusting agent is sodium chloride. Typically, the concentration of sodium chloride suitable for use in the pharmaceutical compositions is between about 0.1% (w/v) to about 1.8%. By way of example, suitable sodium chloride concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8% (which is equivalent to 8 mg/ml), from out 0.8% to about 0.9% (which is equivalent to 9 mg/ml), from about 0.9% to about 1.0%, from about 1% to about 1.2%, from 1.2% (which is equivalent to 12 mg/ml) to about 1.4%, from about 1.4% to about 1.6%, and from about 1.6% to about 1.8%.

In some embodiments, the pharmaceutical compositions comprise two, three, four, or more tonicity adjusting agents. In these embodiments, the concentration of each tonicity adjusting agent is typically less than the concentration that is used when only a single agent is present in the formulation.

Buffers when used in the formulations of the present invention include, but are not limited to, pharmaceutically acceptable salts and acids of acetate, glutamate, citrate, tartrate, benzoate, lactate, histidine or other amino acids such as arginine, alanine, glycine and lysine, gluconate, phosphate, malate, succinate, formate, propionate, and carbonate. “Pharmaceutically acceptable” is used herein in the sense of being compatible with the other ingredients of the formulation and not deleterious to the recipient thereof. Accordingly, the term “pharmaceutically acceptable salt” references salt forms of the active compounds which are prepared with counter ions which are non-toxic under the conditions of use and are compatible with a stable formulation. Buffers suitable for use in the formulations of the present invention also include, but are not limited to tris (hydroxymethyl)aminomethane (TRIS), triethanolamine, trolamine, diethanolamine, meglumine etc.

The concentration of the buffer in the formulation can be expressed in mg/ml, g/l or as a molar concentration. Typically, from about 0.0001 mg/ml to about 100 mg/ml of a suitable buffer is present in the formulations of the present invention. Thus, the formulations can comprise from about 0.0001 to about 0.001 mg/ml of a suitable buffer, from about 0.001 to about 0.01 mg/ml of a suitable buffer, from about 0.01 to about 0.1 mg/ml of a suitable buffer, from about 0.1 to 1 mg/ml of a suitable buffer, from about 1 to about 5 mg/ml of a suitable buffer, from about 5 to about 10 mg/ml of a suitable buffer, from about 10 to about 15 mg/ml of a suitable buffer, from about 15 to about 20 mg/ml of a suitable buffer, from about 20 to about 25 mg/ml of a suitable buffer, from about 25 to about 50 mg/ml of a suitable buffer, from about 50 to about 75 mg/ml of a suitable buffer, and from about 75 to about 100 mg/ml of a suitable buffer.

Alternatively, the buffer concentration can be expressed as molar concentrations. In typical embodiments, from about 0.1 to 100 mM of a suitable buffer is present in the pharmaceutical compositions. Thus, the pharmaceutical compositions can comprise a suitable buffer having a concentration from about 0.1 to about 100 mM, from about 0.1 to about 0.5 mM, from about 0.5 to about 1.0 mM, from about 1.0 to about 5 mM, from about 5 to about 10 mM, from about 10 to about 15 mM, from about 15 to about 25 mM, from about 25 to about 50 mM, from about 50 to about 75 mM, and from about 75 to about 100 mM.

In some embodiments, the formulations of the present invention further comprise a pH adjusting agent. Suitable pH adjusting agents typically include at least an acid or a salt thereof, and/or a base or a salt thereof. Acids and bases can be added on an as needed basis in order to achieve the desired pH. For example, if the pH is greater than the desired pH, an acid can be used to lower the pH to the desired pH. Acids suitable for use in formulations include, but are not limited to, hydrochloric acid, phosphoric acid, citric acid, ascorbic acid, acetic acid, sulphuric acid, carbonic acid, nitric acid and the like. By way of another example, if the pH is less than the desired pH, a base can be used to adjust the pH to the desired pH. Bases suitable for use in formulations include, but are not limited to, sodium hydroxide, potassium hydroxide, calcium hydroxide, sodium carbonate, sodium citrate, sodium acetate, magnesium hydroxide and the like.

A “Buffering agent” or a “pH adjusting agent” as used herein is a system which is used for the purposes and is capable of maintaining the desired/required pH of the formulations throughout desired/required time period, e.g. stability studies and/or shelf life of the drug product. The desired pH of the formulations according to the present invention is between about 6.0 and about 11.0.

In some of the embodiments of the invention, both buffering agent and pH adjusting agent are used. In some of the embodiments of the invention, only a buffering agent is used. In some of the embodiments of the invention, only a pH adjusting agent is used.

In some of the preferred embodiments, sodium hydroxide is used as a pH adjusting agent which adjusts and/or maintains the desired/required pH.

Microbiological contamination presents a significant health hazard in liquid formulations. Therefore, the use of preservatives become inevitable to prevent the growth of microorganisms during the product's manufacture and shelf life, although it may be most desirable to develop a “preservative-free” formulation to address the increasing concerns about the biological activity of these compounds. Most formulations require some kind of preservative to ensure no microbial growth. Non-limiting examples of preservatives when used in the formulations of the present invention include Alcohol, Ethanol, Chlorobutanol, Phenoxyethanol, Potassium benzoate, Benzyl alcohol, Benzoic acid, Potassium sorbate, Sorbic acid, Benzalkonium chloride, Benzethonium chloride, Cetrimonium bromide, Cetylpyridinium chloride, Bronopol, Chlorbutol, Chlorocresol, Cresol, Butylparaben, Methylparaben, Propylparaben, Ethylparaben, Phenol, Thymol, Phenylethanol, Sodium benzoate, Antimicrobial solvents like Propylene glycol, Glycerin, Chloroform and the like.

Solubilizing agents or solvents or co-solvents when used in the formulations of the present invention without limitation include dichloromethane, acetonitrile, ethyl acetate, acetone, propylene carbonate, water, glycerine, coconut fatty acid diethanolamide, medium and/or long chain fatty acids or glycerides, monoglycerides, diglycerides, triglycerides, structured triglycerides, soyabean oil, peanut oil, corn oil, corn oil monoglycerides, corn oil diglycerides, corn oil triglycerides, polyethylene glycol, caprylocaproylmacroglycerides, caproyl 90, propylene glycol, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, acetone, methyl isobutyl ketone, methyl ethyl ketone and any combination thereof.

Surfactants when used in the formulations of the present invention without limitation include sodium lauryl sulfate, docusate sodium, cocamidopropyl amino betaine, polyoxyethylene sorbitan fatty acid esters (Polysorbate, Tween®), polyoxyethylene 15 hydroxystearate (Macrogol 15 hydroxystearate, Solutol HS 15®), polyoxyethylene castor oil derivatives (Cremophor® EL, ELP, RH 40), polyoxyethylene stearates (Myrj®), sorbitan fatty acid esters (Span®), Polyoxyethylene alkyl ethers (Brij®), polyoxyethylene nonylphenol ether (Nonoxynol®) and a combination thereof.

Stabilizing agents when used in the formulations of the present invention without limitation include salts of iodide, sodium metabisulphite, sodium bisulphite, ethylene diamine tetraacetic acid (EDTA) or salts thereof, ascorbic acid, povidone, povidone K12, povidone K17, crospovidone, sorbitol, sorbitol solution and a combination thereof.

The pharmaceutically acceptable salt of diclofenac comprises any soluble salt of diclofenac with a pharmaceutically acceptable organic or inorganic base. Non-limiting examples of pharmaceutically acceptable inorganic bases are hydroxides, carbonates and hydrogen carbonates of ammonium, calcium, magnesium, sodium and potassium, for instance sodium hydroxide, ammonium hydroxide, calcium hydroxide, magnesium carbonate, sodium hydrogen carbonate and potassium hydrogen carbonate. Non-limiting examples of pharmaceutically acceptable organic bases are arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, N-methylglucamine, glucamine, glucosamine, histidine, N-(2-hydroxyethyl)piperidine, N-(2-hydroxyethyl)pyrrolidine (epolamine salt), isopropylamine, lysine, methylglucamine, morpholine, piperazine, piperidine, theobromine, triethylamine, trimethylamine, tripropylamine and tromethamine. The preferred Diclofenac salt is Diclofenac epolamine. The amount of Diclofenac or its salt present in the formulations of the present invention varies between about 10 mg and about 200 mg. The amount of Diclofenac epolamine varies between 24.17 to 48.34 mg in 0.5 to 1 ml solutions respectively, and more preferably 1 ml solutions.

The formulations of the present invention can be administered by intravenous bolus injection over 15 seconds every 6 hours as needed, not to exceed 193.36 mg/day.

The invention provides a composition comprising Diclofenac epolamine in a concentration of 24.17-48.34 mg for parenteral through intramuscular, intravenous, subcutaneous, intra-articular route. The composition comprises Diclofenac epolamine, an antioxidant, a tonicity adjusting agent, a pH adjusting agent and water for injection.

### Definitions:

The use of the terms “a” and “an” and “the” and similar referents in the context of describing the invention are to be construed to cover both the singular and the plural, unless otherwise indicated herein or clearly contradicted by context.

As used herein, the term “about” is synonymous with “approximately” and is used to provide flexibility to a numerical value or range endpoint by providing that a given value may be “a little above” or “a little below” the value stated. “About” can mean, for example, within 3 or more than 3 standard deviations. “About” can mean within a percentage range of a given value. For example, the range can be ±1%, ±5%, ±10%, ±20%, ±30%, ±40% or ±50% of a given value. “About” can mean with an order of magnitude of a given value, for example, within 2-fold, 3-fold, 4-fold or 5-fold of a value. However, it is to be understood that even when a numerical value is accompanied by the term “about” in this specification, that express support shall be provided at least for the exact numerical value as well as though the term “about” were not present.

As used herein, the terms “stable” or “stability” encompass any characteristic of the formulations which may be affected by storage conditions including, without limitation, potency, total impurities, degradation products, specific optical rotation, optical purity, water content, appearance, viscosity, sterility, and colour and clarity. The storage conditions which may affect stability include, for example, duration of storage, temperature, humidity, and/or light exposure.

The term “degradant”, “impurity”, “degradation impurity” and “related substance” as used herein represents the same meaning and can be used interchangeably.

In some embodiments, the formulations of the present invention are stable for prolonged time when stored under storage conditions. The term “storage conditions” as used herein without limitation include typical storage conditions such as 2° C.-8° C. The compositions of the present invention may also be stored at 40° C.±2° C./75±5% RH, 30° C.±2° C./65±5% RH, 25° C.±2° C./40±5% RH, 25° C.±2° C./60±5% RH, 40° C.±2° C./NMT 25% RH (NMT=not more than) and accelerated conditions such as 40° C.±2° C./75±5% RH. The term “prolonged time” as used herein indicates that the formulations of the present invention are stable for at least 1 month or more, at least 3 months or more, at least 6 months or more or at least 12 months or more when stored under storage conditions.

In some of the embodiments of the present invention, “stable” or “storage stable”, or “stability” when used with reference to the formulations of the present invention or when used “stable or storage stable formulations” or “stability of the formulations” all these terms/phrases refer to formulations of the present invention which retain at least about 90%, or at least about 95%, or at least about 96%, or at least about 98%, of the labelled concentration of Diclofenac or salt thereof contained in the said formulation after storage under typical and/or accelerated conditions. In further embodiments, stable formulations or stability of the formulations refer to less than about 15% (area percent), or less than about 10% (area percent), or less than about 7% (area percent), or less than about 5% (area percent), or less than about 2% (area percent) of Diclofenac-related impurities are present after storage under typical and/or accelerated conditions.

In some of the embodiments, formulations of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) any known or unknown single Diclofenac-related impurity or other impurity after storage under typical and/or accelerated conditions.

In some of the embodiments, formulations of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) total Diclofenac-related impurities or other impurities after storage under typical and/or accelerated conditions.

Methods for determining the stability of the formulations of the present invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of any individual impurities (known/unknown), or total impurities reported herein in the formulations are determined by a peak area percent method using HPLC.

The term “compatible” as used herein refers to those added excipients or ingredients or additives those are not substantially antagonistic to the other excipients or ingredients or additives or pharmaceutically active ingredients.

As used herein, “treatment” refers to ameliorating or reducing symptoms associated with a disease or condition. Treatment means any manner in which the symptoms of a condition, disorder or disease are ameliorated or otherwise beneficially altered. Hence treatment encompasses prophylaxis, therapy and/or cure. Treatment also encompasses any pharmaceutical use of the compositions herein.

As used herein, “treating” a subject having a disease or condition means that a composition or other product provided or described herein is administered to the subject to thereby effect treatment thereof.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “prevention” or “prophylaxis” refers to methods in which the risk of developing disease or condition is reduced. Prophylaxis includes reduction in the risk of developing a disease or condition and/or a prevention of worsening of symptoms or progression of a disease, or reduction in the risk of worsening of symptoms or progression of a disease.

As used herein an “effective amount” of a compound or composition for treating a particular disease is an amount that is sufficient to ameliorate, or in some manner reduce symptoms to achieve the desired physiological effect. Such amount can be administered as a single dosage or can be administered according to a regimen, whereby it is effective. The effective amount is readily determined by one of skill in the art following routine procedures.

As used herein, “disease” or “disorder” or “condition” refers to a pathological condition in an organism resulting from cause or condition including, but not limited to, infections, acquired conditions, genetic conditions, and characterized by identifiable symptoms.

As used herein, “patient” or “subject” to be treated includes humans and or non-human animals, including mammals. Mammals include primates, such as humans, chimpanzees, gorillas and monkeys; domesticated animals, such as dogs, horses, cats, pigs, goats, cows; and rodents such as mice, rats, hamsters and gerbils.

As used herein, “optional” or “optionally” means that the subsequently described event or circumstance does or does not occur, and that the description includes instances where said event or circumstance occurs and instances where it does not. For example, an optionally substituted group means that the group is un-substituted or is substituted.

As used herein, “comprises”, “comprising”, “containing” and “having” and the like can have the meaning ascribed to them in patent law and can mean “includes”, “including” and the like, and are generally interpreted to be open ended terms. The terms “consisting of” or “consists of” are closed terms, and include only the components, structures, steps, or the like specifically listed in conjunction with such terms, as well as that which is in accordance with patent law. “Consisting essentially of” or “consists essentially of” have the meaning generally ascribed to them by patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising” or “including” it is understood that direct support should be afforded also to “consisting essentially of” language as well as “consisting of” language as if stated explicitly and vice versa. In essence, use of one of these terms in the specification provides support for all of the others. The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

In some embodiments, the present invention also provides a container comprising a composition comprising Diclofenac or its pharmaceutically acceptable salt. In certain embodiments, the container is a vial, an ampoule, a bag, a bottle, a cartridge, or a syringe. In some embodiments, the container, the composition, or both the container and the composition are sterile. Preferably, the container is sealed by way of a closure, such as a stopper, plunger, and/or tip-cap.

The container and closure can be made of glass, plastic, and/or rubber. One or more surfaces of the container and/or closure can be treated with a compound to limit reactivity with one or more components of the formulation. In some embodiments, the container and/or closure are treated with silicon. In other embodiments, the container is treated with ammonium sulfate ((NH₄)₂SO₄). The container can be clear or opaque, and can be any color. In some embodiments, the container is flint colored. In other embodiments, the container is amber colored.

In some embodiments, the invention provides a pre-filled syringe containing a composition of the invention described herein. In some embodiments, a syringe according to the invention is a component of an auto-injector.

All percentages mentioned herein, unless otherwise indicated, are on a w/v basis, i.e.

percentage ingredient (active/inactive) present by weight in the total volume of the composition.

The present invention is further exemplified by the following non-limiting examples.

### BEST MODE OF CARRYING OUT THE INVENTION

### EXAMPLES

The compositions of the present invention are explained in more detail with reference to the following examples. These examples are provided by way of illustration only and should not be construed as to limit the scope or spirit of the claims in any manner.

### Example 1

### Preparation of Diclofenac Injectable Formulation of the Present Invention

| Sr. No. | Name of ingredient | Role of ingredient | mg/ml |
|---|---|---|---|
| 1 | Diclofenac epolamine | Active ingredient | 48.34 |
| 2 | Sodium chloride | Tonicity modifying agent | 5.5 |
| 3 | Monothioglycerol | Antioxidant | 5.0 |
| 4 | Sodium hydroxide | pH adjusting agent | Q.S.* |
| 5 | Water for injection | Vehicle | Q.S.^{#} |

Q.S.* = quantity sufficient to adjust/maintain pH between about 6.0 and 11.0;
Q.S.^{#} = quantity sufficient to 1 ml

### Process of Preparation:

a. Mix and dissolve required quantities of sodium chloride and monothioglycerol in water for injection in a suitable vessel;

b. Sufficient quantity of sodium hydroxide or its aqueous solution is added in step (a) to adjust the pH between about 8.0 and about 10.0;

c. Add and dissolve required quantity of Diclofenac epolamine in step (b); and

d. Adjust the volume of step (c) using water for injection to desired batch size.

The formulation prepared according to the above mentioned process may be sterilized using any suitable process known in the art.

### Example 2

### Stability Studies of the Formulations of the Present Invention

The formulations of the present invention were tested for its stability at various conditions such as 25° C./65% RH, 40° C./75% RH and 30° C./65% RH. The samples were tested after 3 months and the results are summarized in the table below.

| | | 25° C./ | 40° C./ | 30° C./ |
|---|---|---|---|---|
| Test parameter | Initial | 65% RH | 75% RH | 65% RH |
| Description | A clear | A clear | A clear | A clear |
| | colourless | colourless | colourless | colourless |
| | solution | solution | solution | solution |
| pH | 8.39 | 8.41 | 8.42 | 8.35 |
| Osmolality | 297 | 299 | 300 | 305 |
| Assay of Diclofenac | 100.70% | 101.00% | 99.10% | 99.70% |
| epolamine | | | | |
| Assay of | 101.50% | 75.30% | 73.90% | 78.90% |
| monothioglycerol | | | | |

Related substances

| | | | | |
|---|---|---|---|---|
| 2,6-dichlorophenyl- | ND | 0.03% | 0.25% | 0.08% |
| 2-indolinone | | | | |
| Unknown impurity | ND | 0.01% | 0.03% | 0.01% |
| Total impurities | 0.00% | 0.04% | 0.29% | 0.09% |

From above data, it can be seen that the formulations of the present invention are stable when stored under different storage conditions for prolonged time.

### Example 3

### Preparation of Diclofenac Injectable Formulations of the Present Invention

| | Concentration (mg/mL) | | | | | | |
|---|---|---|---|---|---|---|---|
| | Monothioglycerol concentration variation | | | | | | |
| Ingredient(s) | F-1 | F-2 | F-3 | F-4 | F-5 | F-6 | F-7 |
| Diclofenac epolamine | 48.34 | 48.34 | 48.34 | 48.34 | 48.34 | 48.34 | 48.34 |
| Monothioglycerol | 2.5 | 3.5 | 5.0 | 6.5 | 7.5 | 9.0 | 10.0 |
| Sodium chloride | 5.5 | 5.5 | 5.5 | 5.5 | 5.5 | 5.5 | 5.5 |
| Sodium hydroxide | Q.S. to | Q.S. to | Q.S. to | Q.S. to | Q.S. to | Q.S. to | Q.S. to |
| | pH 8.5 | pH 8.5 | pH 8.5 | pH 8.5 | pH 8.5 | pH 8.5 | pH 8.5 |
| Water for injection | Q.S. to | Q.S. to | Q.S. to | Q.S. to | Q.S. to | Q.S. to | Q.S. to |
| | 1 mL | 1 mL | 1 mL | 1 mL | 1 mL | 1 mL | 1 mL |

Q.S. = quantity sufficient

| | Concentration (mg/mL) | | | | |
|---|---|---|---|---|---|
| | Sodium chloride concentration variation | | | | |
| Ingredient(s) | F-8 | F-9 | F-10 | F-11 | F-12 |
| Diclofenac epolamine | 48.34 | 48.34 | 48.34 | 48.34 | 48.34 |
| Monothioglycerol | 5.0 | 5.0 | 5.0 | 5.0 | 5.0 |
| Sodium chloride | 4.0 | 5.0 | 5.5 | 6.0 | 6.5 |
| Sodium hydroxide | Q.S. to | Q.S. to | Q.S. to | Q.S. to | Q.S. to |
| | pH 8.5 | pH 8.5 | pH 8.5 | pH 8.5 | pH 8.5 |
| Water for injection | Q.S. to | Q.S.to | Q.S. to | Q.S. to | Q.S. to |
| | 1 mL | 1 mL | 1 mL | 1 mL | 1 mL |

Q.S. = quantity sufficient

| | Concentration (mg/mL) | | |
|---|---|---|---|
| | pH variation | | |
| Ingredient(s) | F-13 | F-14 | F-15 |
| Diclofenac epolamine | 48.34 | 48.34 | 48.34 |
| Monothioglycerol | 5.0 | 5.0 | 5.0 |
| Sodium chloride | 5.5 | 5.5 | 5.5 |
| Sodium hydroxide | Q.S. to pH 8.0 | Q.S. to pH 8.5 | Q.S. to pH 9.0 |
| Water for injection | Q.S. to 1 mL | Q.S. to 1 mL | Q.S. to 1 mL |

Q.S. = quantity sufficient

### Example 4

### Stability Studies of the Formulations of the Present Invention

Formulation: 48.34 mg/mL Diclofenac epolamine; 5.0 mg/mL monothioglycerol;
5.5 mg/mL sodium chloride; sodium hydroxide in a quantity sufficient to
adjust pH 8.5; and water for injection in a quantity sufficient to 1.0 mL

| | | 40° C./75% RH | | | 25° C./60% RH | | | | |
|---|---|---|---|---|---|---|---|---|---|
| | | 1 M | 3 M | 6 M | 1 M | 3 M | 6 M | 9 M | 12 M |
| Test | Initial | Inverted | Inverted | Inverted | Inverted | Inverted | Inverted | Inverted | Inverted |
| Description | Clear | Clear | Clear | Clear | Clear | Clear | Clear | Clear | Clear |
| | colorless | colorless | colorless | colorless | colorless | colorless | colorless | colorless | colorless |
| | solution | solution | solution | solution | solution | solution | solution | solution | solution |
| % assay of | 103.2 | 102.6 | 103.5 | 102.9 | 102.8 | 103.7 | 102.8 | 103.0 | 102.4 |
| diclofenac | | | | | | | | | |
| % assay of | 62.2 | 69.7 | 86.5 | 66.0 | 83.2 | 88.4 | 84.0 | 84.3 | 82.1 |
| monothioglycerol | | | | | | | | | |

Related substances (%)

| | | | | | | | | | |
|---|---|---|---|---|---|---|---|---|---|
| 2,6- | 0.04 | 0.12 | 0.31 | 0.62 | 0.03 | 0.03 | 0.06 | 0.1 | 0.13 |
| dichlorophenyl- | | | | | | | | | |
| 2-indolinone | | | | | | | | | |
| Single maximum | 0.01 | 0.01 | 0.01 | 0.02 | ND | ND | 0.01 | 0.01 | ND |
| unknown | ND | ND | ND | ND | ND | ND | ND | ND | 0.01 |
| impurity | 0.01 | 0.01 | ND | 0.01 | ND | ND | 0.01 | 0.01 | ND |
| | ND | ND | ND | ND | ND | ND | ND | ND | 0.01 |
| | ND | 0.01 | ND | ND | ND | ND | ND | ND | ND |
| | ND | ND | ND | ND | ND | ND | ND | ND | ND |
| | 0.01 | 0.01 | 0.01 | ND | ND | ND | ND | ND | ND |
| Total impurities | 0.07 | 0.16 | 0.33 | 0.65 | 0.03 | 0.03 | 0.08 | 0.12 | 0.15 |
| pH | 8.54 | 8.51 | 8.57 | 8.57 | 8.46 | 8.5 | 8.46 | 8.52 | 8.48 |

ND = not detected;
RH = relative humidity

### Example 5

### Stability Studies of the Formulations of the Present Invention

Formulation: 48.34 mg/mL Diclofenac epolamine; 2.5 mg/mL monothioglycerol;
5.5 mg/mL sodium chloride; sodium hydroxide in a quantity sufficient to
adjust pH 8.5; and water for injection in a quantity sufficient to 1.0 mL

| | | 40° C./75% RH | | | 25° C./60% RH | | | |
|---|---|---|---|---|---|---|---|---|
| | | 1 M | 3 M | 6 M | 1 M | 3 M | 6 M | 9 M |
| Test | Initial | Inverted | Inverted | Inverted | Inverted | Inverted | Inverted | Inverted |
| Description | Clear | Clear | Clear | Clear | Clear | Clear | Clear | Clear |
| | colorless | colorless | colorless | colorless | colorless | colorless | colorless | colorless |
| | solution | solution | solution | solution | solution | solution | solution | solution |
| % assay of | 102 | 101.2 | 101.7 | 99.6 | 102.2 | 102.0 | 101.6 | 103.9 |
| diclofenac | | | | | | | | |
| % assay of | 91.1 | 85.6 | 79.4 | 66.1 | 87.5 | 86.7 | 79.4 | 72.4 |
| monothioglycerol | | | | | | | | |

Related substances (%)

| | | | | | | | | |
|---|---|---|---|---|---|---|---|---|
| 2,6- | ND | 0.11 | 0.28 | 0.52 | 0.01 | 0.03 | 0.06 | 0.09 |
| dichlorophenyl- | | | | | | | | |
| 2-indolinone | | | | | | | | |
| Single maximum | ND | ND | ND | ND | ND | ND | ND | ND |
| unknown | ND | 0.01 | 0.01 | 0.02 | ND | ND | 0.01 | 0.01 |
| impurity | ND | ND | ND | ND | ND | ND | ND | ND |
| | ND | ND | 0.01 | 0.01 | ND | ND | 0.01 | 0.01 |
| | ND | ND | ND | ND | ND | ND | ND | ND |
| | ND | ND | ND | ND | ND | ND | ND | ND |
| | ND | ND | ND | ND | ND | ND | ND | 0.01 |
| Total impurities | ND | 0.12 | 0.30 | 0.55 | 0.01 | 0.03 | 0.08 | 0.12 |
| pH | 8.71 | 9.75 | 8.78 | 8.80 | 8.74 | 8.71 | 8.70 | 8.69 |
| Osmolality | 260 | 265 | 260 | 262 | 257 | 267 | 266 | 259 |

ND = not detected;
RH = relative humidity

The compositions of the present invention as described herein are suitable for use in the industry.

It should be understood that various changes and modifications to the embodiments described herein will be apparent to those skilled in the art. Such changes and modifications can be made without departing from the spirit and scope of the subject matter of the present invention and without diminishing its intended advantages. It is therefore intended that such changes and modifications be covered within the scope of the present invention.

1. A parenteral composition of diclofenac comprising diclofenac epolamine and one or more pharmaceutically acceptable excipients.

2. The parenteral composition of diclofenac as claimed in claim 1, wherein one or more pharmaceutically acceptable excipients selected from an antioxidant, a tonicity adjusting agent, a pH adjusting agent, an aqueous vehicle, a preservative, a chelating agent, a buffering agent, a solubilizing agent or a solvent or a co-solvent, a surfactant, a stabilizing agent and a combination thereof.

3. The parenteral composition of diclofenac as claimed in claim 1, wherein said composition has pH between about 6.0 and about 11.0.

4. The parenteral composition of diclofenac as claimed in claim 2, wherein an antioxidant is selected from the group consisting of butylated hydroxytoluene, butylated hydroxyanisole, tert-butyl-hydroquinone, 4-hydroxymethy-1-2,6-di-tert-butylphenol, 2,4,5-trihydroxybutyrophenone, alkylgallates, propyl gallate, octyl gallate, dodecyl gallate, ethoxyquin, gallic acid, nordihydroguaiaretic acid, glycine, ascorbic acid, fatty acid esters of ascorbic acid, ascorbyl palmitate, ascorbyl stearate, salts of ascorbic acid, erythorbic acid, L-carnitine, thioglycerol, monothioglycerol, acetyl L-carnitine, thioglycolic acid, N-acetyl cysteine or its salts, cysteine or its salts, glutathione, methionine, tartaric acid, citric acid, fumaric acid, glycolic acid, oxalic acid, succinic acid, ellagic acid, malic acid, maleic acid, tocopherols, delta tocopherol, alpha tocopherol, lipoic acid, thiolated polymers, polymethacrylic-SH, carboxy methylcellulose-cysteine, polycarbophil-cysteine, beta-carotene, carotenoids, flavonoids, flavones, isoflavones, flavanones, catechins, anthocyanidins, chalcones, potassium sulfite, sodium metabisulfite, sodium sulfite, sodium thiosulfate, sodium formaldehyde sulfoxylate, sodium bisulfite and a combination thereof;
a tonicity adjusting agent is selected from the group consisting of anhydrous or hydrous forms of sodium chloride, dextrose, sucrose, xylitol, fructose, glycerol, sorbitol, mannitol, potassium chloride, mannose, calcium chloride, magnesium chloride and a combination thereof;
a pH adjusting agent is selected from the group consisting of acetic acid, adipic acid, ammonium carbonate, ammonium hydroxide, boric acid, citric acid, fumaric acid, hydrochloric acid, malic acid, nitric acid, propionic acid, potassium bicarbonate, potassium chloride, sodium bicarbonate, sodium borate, sodium carbonate, sodium chloride, sodium hydroxide, sodium propionate, succinic acid, sulfuric acid, tartaric acid, triethylamine and a combination thereof;
an aqueous vehicle is selected from water for injection;
a preservative is selected from ethanol, chlorobutanol, phenoxyethanol, potassium benzoate, benzyl alcohol, benzoic acid, potassium sorbate, sorbic acid, benzalkonium chloride, benzethonium chloride, cetrimonium bromide, cetylpyridinium chloride, bronopol, chlorbutol, chlorocresol, cresol, butylparaben, methylparaben, propylparaben, ethylparaben, phenol, thymol, phenylethanol, sodium benzoate, propylene glycol, glycerin, chloroform and a combination thereof;
a chelating agent is selected from ethylene diaminetetraacetic acid (EDTA), deferoxannine, desferrioxannine B, dithiocarb sodium, penicillamine, pentetate calcium, a sodium salt of pentetic acid, succimer, trientine, nitrilotriacetic acid, diethylenetriaminepentaacetic acid, trans-diaminocyclohexanetetraacetic acid (DCTA), dihydroethylglycine, bis(anninoethyl)glycolether-N,N,N′,N′-tetraacetic acid, iminodiacetic acid, poly(aspartic acid), citric acid, tartaric acid, fumaric acid, succinic acid, glycolic acid, lactic acid, oxalic acid, malic acid, lecithin or any salt thereof and a combination thereof;
a buffering agent is selected from pharmaceutically acceptable salts and acids of acetate, glutamate, citrate, tartrate, benzoate, lactate, histidine or other amino acids such as arginine, alanine, glycine and lysine, gluconate, phosphate, malate, succinate, formate, propionate, carbonate and tris (hydroxymethyl)aminomethane (TRIS), triethanolamine, trolamine, diethanolamine, meglumine and a combination thereof;
a solubilizing agent or a solvent or a co-solvent is selected from dichloromethane, acetonitrile, ethyl acetate, acetone, propylene carbonate, water, glycerine, coconut fatty acid diethanolamide, medium and/or long chain fatty acids or glycerides, monoglycerides, diglycerides, triglycerides, structured triglycerides, soyabean oil, peanut oil, corn oil, corn oil monoglycerides, corn oil diglycerides, corn oil triglycerides, polyethylene glycol, caprylocaproylmacroglycerides, caproyl 90, propylene glycol, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, acetone, methyl isobutyl ketone, methyl ethyl ketone and a combination thereof;
a surfactant is selected from sodium lauryl sulfate, docusate sodium, cocamidopropyl amino betaine, polyoxyethylene sorbitan fatty acid esters (Polysorbate, Tween®), polyoxyethylene 15 hydroxystearate (Macrogol 15 hydroxystearate, Solutol HS 15®), polyoxyethylene castor oil derivatives (Cremophor® EL, ELP, RH 40), polyoxyethylene stearates (Myrj®), sorbitan fatty acid esters (Span®), Polyoxyethylene alkyl ethers (Brij®), polyoxyethylene nonylphenol ether (Nonoxynol®) and a combination thereof; and
a stabilizing agent is selected from salts of iodide, sodium metabisulphite, sodium bisulphite, ethylene diamine tetraacetic acid (EDTA) or salts thereof, ascorbic acid, povidone, povidone K12, povidone K17, crospovidone, sorbitol, sorbitol solution and a combination thereof.

5. A parenteral composition of diclofenac comprising diclofenac epolamine and a tonicity adjusting agent.

6. The parenteral composition of diclofenac as claimed in claim 5 further comprising one or more excipients selected from an antioxidant, a pH adjusting agent, a vehicle and a combination thereof.

7. The parenteral composition of diclofenac as claimed in claim 5, wherein said composition has pH between about 6.0 and 11.0.

8. The parenteral composition of diclofenac as claimed in claim 5, wherein a tonicity adjusting agent is selected from the group consisting of anhydrous or hydrous forms of sodium chloride, dextrose, sucrose, xylitol, fructose, glycerol, sorbitol, mannitol, potassium chloride, mannose, calcium chloride, magnesium chloride and a combination thereof.

9. The parenteral composition of diclofenac as claimed in claim 6, wherein an antioxidant is selected from the group consisting of butylated hydroxytoluene, butylated hydroxyanisole, tert-butyl-hydroquinone, 4-hydroxymethy-1-2,6-di-tert-butylphenol, 2,4,5-trihydroxybutyrophenone, alkylgallates, propyl gallate, octyl gallate, dodecyl gallate, ethoxyquin, gallic acid, nordihydroguaiaretic acid, glycine, ascorbic acid, fatty acid esters of ascorbic acid, ascorbyl palmitate, ascorbyl stearate, salts of ascorbic acid, erythorbic acid, L-carnitine, thioglycerol, monothioglycerol, acetyl L-carnitine, thioglycolic acid, N-acetyl cysteine or its salts, cysteine or its salts, glutathione, methionine, tartaric acid, citric acid, fumaric acid, glycolic acid, oxalic acid, succinic acid, ellagic acid, malic acid, maleic acid, tocopherols, delta tocopherol, alpha tocopherol, lipoic acid, thiolated polymers, polymethacrylic-SH, carboxy methylcellulose-cysteine, polycarbophil-cysteine, beta-carotene, carotenoids, flavonoids, flavones, isoflavones, flavanones, catechins, anthocyanidins, chalcones, potassium sulfite, sodium metabisulfite, sodium sulfite, sodium thiosulfate, sodium formaldehyde sulfoxylate, sodium bisulfite and a combination thereof;
a pH adjusting agent is selected from the group consisting of acetic acid, adipic acid, ammonium carbonate, ammonium hydroxide, boric acid, citric acid, fumaric acid, hydrochloric acid, malic acid, nitric acid, propionic acid, potassium bicarbonate, potassium chloride, sodium bicarbonate, sodium borate, sodium carbonate, sodium chloride, sodium hydroxide, sodium propionate, succinic acid, sulfuric acid, tartaric acid, triethylamine and a combination thereof; and
a vehicle selected from water for injection;

10. A parenteral composition of diclofenac comprising:
about 10 mg to about 200 mg diclofenac epolamine;
sodium chloride;
monothioglycerol;
sodium hydroxide; and
water for injection.
